(19) United States
(12) Patent Application Publication
(10) Pub. No.: US 2025/0101474 A1
(43) Pub. Date: Mar. 27, 2025

(54) NATURAL EXTRACTION OF CANNABIS-BASED PHYTOCOMPOUNDS FROM CANNABIS PLANTS THROUGH YEAST FERMENTATION

(71) Applicant: SAN MARTIN CBD LLC, San Mateo, CA (US)

(72) Inventor: Steve TSURUDOME, San Mateo, CA (US)

(21) Appl. No.: 18/833,855

(22) PCT Filed: Jan. 27, 2023
(86) PCT No.: PCT/US2023/061486

§ 371 Date: Jul. 26, 2024

Related U.S. Application Data
(60) Provisional application No. 63/304,518, filed on Jan. 28, 2022.

(51) Int. Cl. C12P 17/06; A23L 2/38; A23L 33/105; A23L 33/14; C12N 1/18; C12R 1/865

(52) U.S. Cl. CPC C12P 17/06; A23L 2/382; A23L 33/105; A23L 33/14; C12N 1/185; C12R 2001/865

(57) ABSTRACT

A fermentation process for extracting cannabis-based phytocompounds is described. The process utilizes wine or bear yeasts to ferment cannabis flowers, resulting in cannabis lees enriched with cannabis-based phytocompounds.

### BACKGROUND

### Technical Field

This disclosure relates to the natural extraction of cannabis-based phytocompounds (e.g., cannabinoids, terpenes, and phenolic compounds) from fresh and dried cannabis plants through yeast fermentation.

### Description of the Related Art

Cannabis plants, including marijuana and hemp, contain various amounts of cannabinoids like cannabidiol (CBD), cannabigerol (CBG), and tetrahydrocannabinol (THC), which are known to have beneficial psychoactive and medicinal effects. Other active components such as terpenoids and flavonoids are known to synergistically contribute to these beneficial effects, in addition to providing flavors. The complex synergy between all of the different components of cannabis is also known as the entourage effect.

The cannabinoids from the cannabis plants (especially the flowers) may be consumed by inhaling (e.g., smoking the whole plant or vaping oils), or by ingestion or sublingual absorption of the plant extracts in edibles or tinctures. While edibles and tinctures contain a number of cannabinoid extracts, they rarely achieve the full and broad-spectrum effects or flavors of the whole plant. Cannabinoid extracts are typically obtained through various extraction methods which include super critical CO₂, alcohol extraction, and butane or propane extraction, among others. However, some volatile or reactive compounds, including the flavorful terpenes, can be degraded and lost under the extraction conditions (e.g. through evaporation) or chemically changed (e.g., degradation) to different species.

Smoking thus remains the most common and preferred method of consuming the cannabinoids through the whole plant approach. There is a need, however, to develop natural, in particular, whole plant preparations that do not involve heating and does not involve harsh industrial chemical extraction methods.

### BRIEF SUMMARY

Disclosed herein are natural extraction methods that capture a full spectrum of active phytocompounds present in whole cannabis flowers, thus delivering certain beneficial effects of whole plants without inhalation or involving harsh industrial chemical extraction methods. In particular, the process comprises step-wise fermentation of cannabis (e.g., whole cannabis flowers), resulting in one or more cannabinoids or other phytocompounds being extracted from the cannabis and enriched within “cannabis lees,” a naturally formed suspension medium comprising fermentation by-products such as dead yeast cells.

Thus, one embodiment provides a process for extracting cannabis-based phytocompounds, the process comprising: providing a yeast starter culture comprising Saccharomyces, sugar and water; allowing the yeast starter culture to ferment for a first period sufficient to provide a first fermentation medium, whereby the Saccharomyces propagate; adding cannabis to the first fermentation medium to provide a second fermentation medium; and allowing for the second fermentation medium to ferment for a period to provide cannabis lees comprising cannabis-based phytocompounds.

### BRIEF DESCRIPTION OF THE SEVERAL VIEWS OF THE DRAWINGS

Exemplary embodiments are illustrated in referenced figures. It is intended that the embodiments and figures disclosed herein are to be considered illustrative rather than restrictive.

FIG. 1 depicts a flow diagram providing an overview of the fermentation process according to an embodiment.

FIG. 2 shows a container of fermented hemp flowers, in which cannabis lees formed at the bottom of the container.

### DETAILED DESCRIPTION

Disclosed herein is a process that combines fermentation and extraction to provide extracted cannabis-based phytocompounds. The process preserves the entourage effect of consuming the whole plant by extracting a spectrum of phytocompounds such as cannabinoids, flavonoids and terpenes naturally present in a given strain of cannabis, delivering an entourage effect with synergistic benefits to the consumer of the resulting fermented beverage.

Advantageously, the fermentation process produces ethanol, which acts as an extraction medium to extract the phytocompounds from the cannabis plants, preferably, whole flowers. Furthermore, as the fermentation proceeds, cannabis lees are formed. Cannabis lees are suspensions of byproducts of the fermentation process, the byproducts including complex natural molecules (e.g., proteins, fatty acids, polysaccharides, enzymes, etc) from dead or broken down yeast cells. Cannabis lees are surprisingly found to have significant solubilizing and emulsifying capabilities. The ethanol-extracted phytocompounds are enriched and co-suspended in the cannabis lees, which are readily separable from the rest of the fermentation broth.

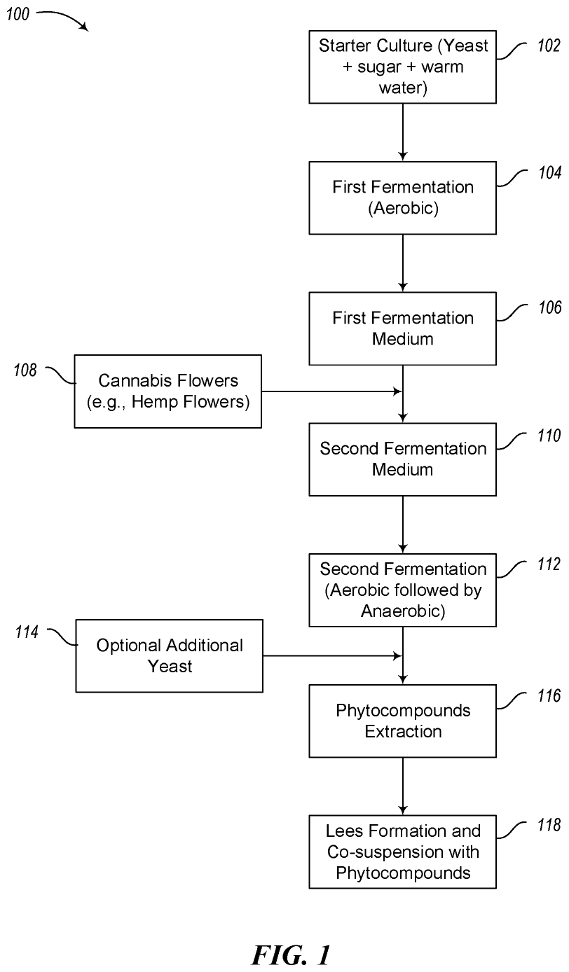

As illustrated in FIG. 1, the natural extraction process (100) begins with a first fermentation step where Saccharomyeces (e.g., Saccharomyces cerevisiae) is rehydrated and activated with water and sugar to create a yeast starter culture (102). The first fermentation step (104) causes the propagation of yeast cells and an initial production of ethanol, resulting in a first fermentation medium (106). Typically, the first fermentation step takes place aerobically to produce a robust yeast starter culture. Ethanol is also produced as a product of the fermentation. Typically, the ethanol content is less than 5% v/v, or more typically, less than 3% v/v of the first fermentation medium. In some embodiments, the initial ethanol content is 0.1-5% v/v, or more typically, 1-3% v/v.

Cannabis flowers (108) are then added to the first fermentation medium (106) to provide a second fermentation medium (110). During the second fermentation step, ethanol is continuously produced, which provides an extraction medium. During fermentation, the ethanol content may be as high as 8%, and more typically may be up to 5%. Cannabis (including fresh, dried or cured flower, or decarboxylated flowers) are fermented under conditions first aerobically and then anaerobically (112) for sufficient time to allow for the natural extraction (116) of full and broad spectrum cannabis-based phytocompounds.

Optionally, additional Saccharomyeces (Saccharomyces cerevisiae) (114) may be added to the second fermentation medium, which may have a starting ethanol content of 1-3%. The additional brewer's yeast invigorates the alcoholic fermentation and increases the ethanol content, e.g., to 4.5%-8% (v/v). Higher ethanol contents increase the efficiency of the extraction of the terpenoids and cannabinoids from the cannabis.

As the second fermentation step proceeds, cannabis lees are formed and collecting at the bottom of the container. Commonly present in wine-making, lees are deposits of dead yeast cells and other particles that precipitates to the bottom of the vessel at the end of fermentation. Similarly, “cannabis lees,” as used herein, refers to cannabis fermentation byproducts such as the dead or deactivated yeast cells and other fine particles or sediments. Specifically, lees are made up of dead yeast cells, as well as cellular components from the broken down yeast cells, including proteins (e.g., mannoproteins), polysaccharides (glucans), phenolic compounds, enzymes, fatty acids (e.g., phospholipids) and amino acids.

Cannabis lees are thus heterogeneous suspensions with solubilizing and emulsifying capabilities, in which the extracted cannabis phytocompounds are enriched. In some embodiments, the extracted phytocompounds may further undergo enzymatic transformations to provide compounds that are more active or more bioavailable.

Cannabis fermentation thus produces a complex mixture of active cannabis-based phytocompounds (e.g., cannabinoids, terpenes, and phenolic compounds) which are extracted from the cannabis flowers, thereby preserving the entourage effect of consuming the whole plant. Additionally, the second fermentation produces Saccharomyces cerevisiae enzymes which cause enzymatic hydrolysis of the cannabis-based phytocompounds extracted from the cannabis flowers to make these phytocompounds more bioavailable. In particular, the second fermentation decarboxylates approximately 50% of the inactive cannabinoids into bioactive cannabinoids (e.g., inactive CBD-a is found to be decarboxylated into bioactive CBD).

These and other aspects of the disclosure are described in further detail herein.

### First Fermentation Step—Yeast Starter Culture

As used herein, yeast starter culture refers to the actively multiplying yeast cells in water with sugar as food source for the yeast culture.

Suitable yeasts are wine or beer yeasts, including for example, the genus of Saccharomyeces and any one of its species (e.g., S. cerevisiae; S. bayanus; S. boulardii; S. pastorianus; S. kudriavzevii; S. uvarum; S. eubayanus).

In preferred embodiments, Saccharomyces cerevisiae is used. Saccharomyces cerevisiae is a species of yeast most used in fermenting foods and beverages. Also referred to as brewer's yeast or baker's yeast, all strains of Saccharomyces cerevisiae ferment sugar (as a substrate) to produce ethanol and carbon dioxide.

Any type of sugar may be used, including for example, sucrose. Typically, the amount of sugar is about 10-30 times of the yeast (by weight). More typically, the amount of sugar is about 15-20 times of the amount of yeast (by weight).

The yeast starter culture is thus initiated by rehydration of Saccharomyces cerevisiae in warm filtered water (e.g., about 72 to 75 degrees F.). The weight ratio of the yeast and water may be in the range of 1-5% w/v, and more typically, 1-3% w/v.

The starter culture is the first phase in the yeast life cycle, which is concerned with growing the yeast population. Once the yeast cell culture reaches critical mass, alcoholic fermentation gets underway. The yeast starter culture is preferably placed in a dark environment at ambient room temperature (e.g., about 68-78° F.) for 24-48 hours to exponentially increase the yeast population aerobically, with the potential benefit of suppressing harmful bacteria and mold growth.

An initial production of ethanol is possible during the first fermentation step. Typically, the amount of the ethanol is about 0.1-5% v/v at this stage. More typically, the ethanol content is about 0.1-4% v/v, or even more typically, 0.5-4% v/v, 1-4% v/v, 1-3% v/v, 1-2% v/v, and the like.

### Second Fermentation Step—Cannabis Added

After the first fermentation step is carried out for 24-48 hours, cannabis flowers (e.g., dry cured and/or fresh hemp flowers) are added to the yeast starter culture to prepare a second fermentation medium. The amount of cannabis flowers (dry weight) may be 0.5-5 times (by weight), or more typically, 1-3 times, of the amount of the yeast added in the first fermentation step.

After 3 to 5 days of aerobic fermentation, an air-lock stopper may be placed over the vessel containing the yeast starter culture and cannabis flower. The cannabis is then anaerobically fermented with the yeast for 2 to 3 months in a dark environment at ambient room temperature (e.g., about 68-78° F.).

The second fermentation medium undergoes further anaerobic fermentation under conditions and duration optimal for partitioning cannabis extracts into the fermenting broth. As used herein, cannabis extracts may be any compounds that originate from the cannabis plant matter being added. Typically, cannabis flowers (including fresh, cured or decarboxylated flowers) are added because they are enriched with active phytocompounds such as cannabinoids and terpenes.

After the fermentation period of 2 to 3 months, the cannabis-based compounds from the cannabis are extracted and accumulated at the bottom of the vessel forming a tan opaque layer. This tan opaque layer represents the cannabis lees (lees co-suspended with cannabinoid compounds and other cannabis precipitates). Lees are made up of dead yeast cells containing proteins and molecules like mannoproteins, polysaccharides, fatty acids and amino acids. Without wishing to be bound by theories, it is believed that fatty acids and proteins in lees have significant solubilization and emulsification capability, making it possible to extract and enrich lipophilic compounds such as CBD or THC.

### A. Cannabis

Cannabis is a genus of flowering plants in the family Cannabaceae. Unless specified otherwise, cannabis broadly refers to any cultivar within the genus, including marijuana and hemp.

Marijuana is a broad classification of many strains of cannabis containing more than 0.3% (dry weight) of tetrahydrocannabinol (THC), the principal psychoactive constituent. Marijuana (or medical cannabis) is mostly consumed for its constituent cannabinoids, including THC and CBD, in an effort to treat disease or improve symptoms. Cannabis is used to reduce nausea and vomiting during chemotherapy, to improve appetite in people with HIV/AIDS, and to treat chronic pain and muscle spasms. Cannabinoids are under preliminary research for their potential to affect stroke. Although evidence is inconclusive, marijuana has been attempted in treating depression, anxiety, attention deficit hyperactivity disorder, Tourette syndrome, post-traumatic stress disorder, and psychosis.

Certain strains of the cannabis plant are known as hemp, although this term is often used to refer only to varieties of cannabis cultivated for non-drug use (i.e., having 0.3% THC content or less in dry weight). Cannabis has long been used for hemp fiber, hemp seeds and their oils, hemp leaves for use as vegetables and as juice, medicinal purposes, and as a recreational drug. Industrial hemp products are made from cannabis plants selected to produce an abundance of fiber.

Phytocannabinoids include mainly cannabidiol (CBD), tetrahydrocannabinol (THC), cannabigerol (CBG), among the more than 100 cannabinoids identified in cannabis plants. While structurally diverse, cannabinoids all act on the cannabinoid receptors (e.g., CB1 and CB2 receptors), which are located throughout the body and are involved in appetite, pain-sensation, mood, and memory.

Tetrahydrocannabinol or THC is the primary psychoactive cannabinoid component of Cannabis. THC acts on CB1 receptors, which are mostly in the brain, and provides the psychoactive “high” experienced by users. THC is also used as therapies for conditions such as pain, muscle spasticity, glaucoma, insomnia, low appetite, nausea and anxiety.

In cannabis plants, THC occurs mainly as tetrahydrocannabinolic acid (THC-a), which is non-psychoactive. THC-a can be converted to THC by decarboxylation, commonly carried out by heating. However, the fermentation process described herein causes concurrent enzymatic reactions that decarboxylates THC-a to produce THC.

“Cannabidiol” or “CBD” is a non-psychoactive cannabinoid. In combination with THC, CBD demonstrates some modulatory properties to reduce adverse THC effects. CBD is also known to possess its own therapeutic properties, notably for the treatment of seizures and most recently autism. CBD can also benefit those experiencing nausea, inflammation and anxiety due to its antidepressant and neuroprotective effects.

In hemp plants, CBD occurs mainly as cannabidiolic acid (“CBD-a”), the carboxylic acid form of CBD. Like THC-a, CBD-a can also converted to CBD by decarboxylation by enzymatic reaction occurred during fermentation described herein.

Plant terpenes are naturally occurring hydrocarbons of diverse structures. Although terpenes occur in small amounts (less than 5% by weight) of the plant mass, they bring powerful and desirable flavors to the plant extracts. It is also believed that terpenes may modulate the pharmacological effects of cannabinoids.

Phytocannabinoids and terpenes accumulate in the secretory cavity of the glandular trichomes, which largely occur in cannabis flowers (typically of the female plants). Thus, the process disclosed herein preferably utilizes the cannabis flowers, including fresh or cured (dried) flowers. During the second fermentation step, the cannabinoids and terpenes are extracted.

The cannabis flowers may be used cured (dried) by known methods in the art. Alternatively, decarboxylated cannabis flowers may be used. For instance, heating or baking dried cannabis flowers at about 100-120° C. for 60-90 minutes can effectively convert the CBD-a and THC-a to CBD and THC, respectively.

### B. Fermentation Conditions

The second fermentation takes place aerobically for 2 to 3 days, then anaerobically for 2 to 3 months. During the period, the metabolites generated by the yeast form an alcoholic extractant, which selectively partition phytocannabinoids and terpenes from the cannabis flowers. In particular, many cannabinoids have one or two phenol moieties, which can be protonated under alcoholic conditions.

The second fermentation produces Saccharomyces cerevisiae enzymes which causes enzymatic hydrolysis of the cannabis-based phytocompounds extracted from the cannabis flowers to make these hydrophobic phytocompounds more bioavailable. The yeast enzymatic hydrolysis allows for micelle formation from hydrolyzed lipid phytocompounds to aid in bioaccessibility of hydrophobic cannabis-based phytocompounds.

During the second anaerobic fermentation, ethanol is continuously produced by the yeast. While the second fermentation medium begins with an ethanol content of 1-3% (v/v), the ethanol may reach as high as 5-8% (v/v) during the second fermentation step when additional wine/beer yeast is added. The heightened amount of ethanol enhances the extraction of the various active components of the cannabis flowers.

In a more specific embodiments, the cannabis flowers (fresh, cured or decarboxylated) is added at 2-10 g/liter of the second fermentation medium. The fermentation generally takes place in an anaerobic condition at ambient room temperature (e.g., about 68-78° F.) for a sufficient period of time (e.g., 2-3 months).

### Enrichment and Separation of the Extracted Cannabis-based Phytocompounds

The enzymatic hydrolysis of yeast cells at the end of fermentation causes similar enzymatic hydrolysis of nutrient rich cannabis plant cells which then releases the many cannabinoid phytocompounds and becomes emulsified for increased bioavailability. The dead yeast cells and cannabis-based phytocompound precipitates will settle to the bottom of the finished fermentation broth, forming the cannabis lees (lees incorporated with cannabinoid compounds). The cannabis lees contains the cannabis-based phytocompounds of the whole cannabis plant to preserve the entourage effect. This cannabis lees can be analyzed to ascertain the amounts of terpenes and cannabinoids present by known methods in the art.

The cannabis lees can then be titrated into various beverages and edibles by known methods in the art for consumption.

The fermented beverage provides health-benefiting cannabinoids to the beverage (CBD, CBG, etc.). Any psychoactive THC will be reduced to be no more than 0.3% legal limit for Hemp by using Cannabis Hemp flower. The significantly low percentage of THC will produce a beverage that can be consumed by the general public without regulatory restrictions.

The fermented cannabis and hemp flower will add beneficial cannabinoids to the beverage (CBD, CBG, etc.). Any psychoactive THC will be reduced to be no more than 0.3% legal limit for hemp. Advantageously, it is possible to create strain-specific beverages with unique flavor profiles specific to each cannabis strain.

In other embodiments, cannabis flowers that contain a significant percentage of THC, 10% to 20% instead of the low-THC hemp flowers may also be used in the fermentation and THC-enriched cannabis lees are produced.

### EXAMPLES

The disclosure is further illustrated by the following examples. The examples below are non-limiting and are merely representative of various aspects of the disclosure.

### Example 1

### Fermentation of HEMP Flowers

Hemp flowers were fermented according the two-step process disclosed herein.

The yeast starter culture was initiated by rehydrating dried 11.5 g of Saccharomyces cerevisiae (brewer's yeast) with 500 ml of warm filtered water (e.g., 72 to 75° F.) and 220 g of sugar in an Erlenmeyer flask (1000 ml) or similar vessel. Once the yeast reaches critical mass, alcoholic fermentation gets underway. The yeast starter culture was placed in a dark environment at ambient room temperature (e.g., about 68-78° F.) for 24 to 48 hours to increase the yeast population through aerobic fermentation.

After 24 to 48 hours, 10 g of cannabis flowers (dry cured) were added to the yeast starter culture and fermented aerobically for 3 to 5 days. After 3 to 5 days, an air-lock stopper was placed over the Erlenmeyer flask (1000 ml) or similar vessel. The cannabis was fermented anaerobically with the yeast for 2 to 3 months in a dark environment at ambient room temperature (e.g., about 68-78° F.). The flask was periodically shaken to facilitate mixing during this period.

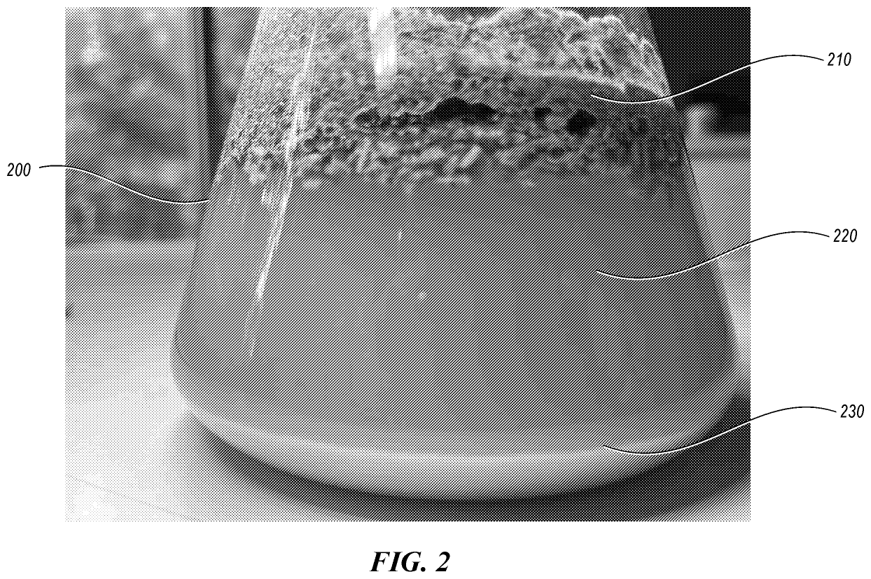

FIG. 2 shows the resulting fermentation products in the Erlenmeyer flask (200) after the second fermentation period of 2 to 3 months. As shown, the remnant hemp flowers (210) remained floating on the top; an aqueous/alcoholic layer (220) was in the middle; and settled to the bottom of the Erlenmeyer flask was a tan opaque layer (230), which was the “cannabis lees.” The cannabis lees layer was separated from the remaining broth by pipetting, which underwent quantitative analysis of its contents.

Table 1 shows the amounts of CBD and CBD-a of fermentation samples taken from the cannabis lees at one-month mark and two-month mark. As shown, not only was CBD-a present in an increasing trend in the cannabis lees, CBD was also formed as a result of decarboxylation.

The aqueous/alcoholic layer was also tested but showed no CBD or CBD-a. This indicates that the phytocannabinoids that were extracted by the ethanol have eventually concentrated in the cannabis lees, which are believed to be more solubilizing than ethanol.

As controls, direct extraction (without fermentation) of CBDs from hemp flowers using water or methyl alcohol were also carried out. As shown in Table 1, water extraction produced only a small amount of CBD-a, but no CBD; whereas the methanol (an industrial chemical) extraction produced a larger amount of the phytocompounds from hemp flowers.

TABLE 1

| | | First | Second | Water | Methyl Alcohol |
|---|---|---|---|---|---|
| | | Month | Month | Control | Control |
| Analyte | LOQ | mg/ml | mg/ml | mg/ml | mg/ml |
| THC-A | 2.1 | ND | ND | ND | ND |
| Δ9-THC | 2.1 | ND | ND | ND | 2.22 |
| CBD-a | 2.1 | 26.9 | 33.1 | 3.09 | 73.2 |
| CBD | 2.1 | 13.9 | 16.5 | ND | 36.4 |
| CBG-a | 2.1 | ND | 2.66 | ND | 6.35 |
| CBG | 2.1 | ND | ND | ND | 3.16 |
| CBN-a | — | — | — | — | — |
| CBN | 2.1 | ND | ND | ND | ND |
| CBC-A | — | — | — | — | — |
| CBC | — | — | — | | — |
| Total | | ND | ND | | 2.22 |
| THC | | | | | |
| Total | | 37.5 | 45.5 | | 100.6 |
| CBD | | | | | |

ND = Not Detected; n/a = Not Analyzed; LOQ = Limit of Quantification; Total THC = (0.877 × THC-A) + Δ9-THC; Total CBD = (0.877 × CBD-A) + CBD.
* Δ8THC has lower precision and higher detection limit than other cannabinoids

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Patent Application No. 63/304,518 filed Jan. 28, 2022, the entirety of which is incorporated by reference herein.

1. A process for extracting cannabis-based phytocompounds, the process comprising:
providing a yeast starter culture comprising Saccharomyeces, sugar and water;
allowing the yeast starter culture to ferment for a first period sufficient to provide a first fermentation medium, whereby the Saccharomyeces propagate;
adding cannabis to the first fermentation medium to provide a second fermentation medium; and
allowing for the second fermentation medium to ferment for a period to provide cannabis lees comprising cannabis-based phytocompounds.

2. The process of claim 1 wherein the Saccharomyces is Saccharomyces cerevisiae.

3. The process of claim 1, wherein the sugar is in an amount of 10-30 times by weight of the Saccharomyeces, or preferably, 15-20 times by weight of the Saccharomyeces.

4. The process of claim 1, wherein the first fermentation medium has an ethanol content of 0.1-5% (v/v).

5. The process of claim 1, wherein first period is about 24-48 hours.

6. The process of claim 1, wherein the yeast starter culture ferments at temperatures of about 72 to 75° F.

7. The process of claim 1, wherein the cannabis comprises flowers of hemps or marijuana.

8. The process of claim 5, wherein the cannabis has a dry weight that is 0.5-5 times (by weight) of the amount of the Saccharomyeces, preferably, the cannabis has a dry weight that is 1-3 times (by weight) of the amount of the Saccharomyeces.

9. The process of claim 1, wherein the second fermentation medium ferments aerobically for up to 5 days followed by fermenting anaerobically for at least 2-3 months.

10. The process of claim 1, wherein the cannabis is hemp and the cannabis lees contain phytocannabinoids including CBD, CBG, CBD-a, or a mixture thereof.

11. The process of claim 1, wherein the cannabis is marijuana and the cannabis lees contain phytocannabinoids including THC, THC-a, or a mixture thereof.

12. The process of claim 1, wherein the cannabis lees further comprises comprise phospholipids.

13. The process of claim 1 further comprising isolating the cannabis lees.

14. A cannabis lee prepared by the process of claim 1.

15. A beverage comprises the cannabis lee of claim 14.

16. A foodstuff comprises the cannabis lee of claim 14.
